(19) United States
(12) Patent Application Publication
(10) Pub. No.: US 2025/0043228 A1
(43) Pub. Date: Feb. 6, 2025

(54) METHODS AND SYSTEMS FOR STERILIZING BIOREACTOR SYSTEMS WITH SUPERHEATED WATER

(71) Applicant: Upside Foods, Inc., Berkeley, CA (US)

(72) Inventor: Konrad Mueller-Auffermann, Emeryville, CA (US)

(21) Appl. No.: 18/741,412

(22) Filed: Jun. 12, 2024

Related U.S. Application Data
(60) Provisional application No. 63/516,794, filed on Jul. 31, 2023.

(51) Int. Cl. C12M 1/12; C12M 3/00

(52) U.S. Cl. CPC C12M 37/00; C12M 21/08

(57) ABSTRACT

The present disclosure relates to systems, apparatuses, and methods for sterilizing a closed bioreactor system utilizing superheated water. In particular, in one or more implementations, the disclosed methods include circulating a heated fluid within at least one pressure vessel of a bioreactor system at a pressure above an atmospheric pressure and a temperature above an atmospheric boiling point for the heated fluid. In some implementations, in response to circulating superheated water within a closed bioreactor system for a predetermined dwell time, the disclosed methods include reducing a pressure within the closed bioreactor system to cause the superheated water to evaporate therein. Also, in one or more implementations, the disclosed systems include a combination superheated water and steam generator, a superheated water storage tank, and/or a stratified energy storage tank for utilization in sterilization-in-place and other procedures for producing comestible cell-based food products.

### CROSS-REFERENCE TO RELATED APPLICATIONS

This application claims priority to and the benefit of U.S. Provisional Patent Application No. 63/516,794, titled “METHODS AND SYSTEMS FOR STERILIZING BIOREACTOR SYSTEMS WITH SUPERHEATED WATER,” filed on Jul. 31, 2023, which is incorporated by reference in its entirety.

### BACKGROUND

As the world's population continues to grow, cell-based or cultured animal products for consumption have emerged as an attractive alternative (or supplement) to conventional slaughtered animal products, such as meat from animals and supplemental products derived therefrom. For instance, cell-based, cultivated, or cultured meat represents a technology that could address the specific dietary needs of humans. Cell-based animal food products can be prepared from one or more of cultivated adherent and suspension cells derived from a non-human animal. Cell-based meat products, for instance, are often formed and shaped to mimic familiar forms of conventional meat.

In addition to addressing dietary needs, cell-based animal food products help alleviate several drawbacks linked to conventional animal products for humans, livestock, and the environment. For instance, conventional meat production involves controversial practices associated with animal husbandry, slaughter, and harvesting. Other drawbacks associated with harvested or slaughtered meat production include low conversion of caloric input to edible nutrients, microbial contamination of the product, emergence and propagation of veterinary and zoonotic diseases, relative natural resource requirements, and resultant industrial pollutants, such as greenhouse gas emissions and nitrogen waste streams.

Despite advances in creating cell-based animal food products, existing methods or systems for cultivating and processing cell-based animal food products face several shortcomings, such as challenges or failures in controlling contamination and other inefficiencies. Existing methods for producing cell-based animal food products are subject to contamination. For example, existing methods often grow and process cells using methods that expose cells to contaminants. Some contaminants can proliferate much faster and survive in less favorable conditions than mammalian, avian, fish, and crustacean cells (e.g., bacteria), which can contaminate and out compete the growth of such cell-based food products.

In avoidance of contamination, for example, existing systems generally utilize steam sterilization to prepare process equipment for cultivation. During sterilization, steam denatures proteins of organisms, thereby reducing the risk of contamination. Producing and implementing clean steam for sterilization, however, presents challenges with cost, efficiency, safety, and equipment maintenance. In particular, the use of steam in conventional systems leads to energy losses, which result from the unavoidable, minor discharge of saturated steam and hot non-condensable gases, which inevitably occur in condensate separators. If the condensate is not reused, water losses occur as well. Furthermore, condensation of saturated steam occurs when the temperature falls below the saturation temperature. In practice, this process results from contact between steam and colder surfaces. Another challenge is the displacement and composition of gases in the containers to be sterilized. Air and CO₂ conduct heat poorly and should be displaced as completely as possible for ideal heat transfer from steam. However, displaying air and CO₂ is not practical for industrial tanks, as it is often too time-consuming and costly. In addition to the foregoing, saturated steam is very aggressive on systems due to its high energy content. During condensation (especially droplet condensation), high amounts of energy are transferred directly to the colder surfaces, which can result in corrosion.

These, along with additional problems and issues persist in existing methods for sterilizing facilities and systems for the production of cell-based animal food products.

### BRIEF SUMMARY

This disclosure generally describes systems, apparatuses, and methods for generating and utilizing superheated water to sterilize process equipment in a closed bioreactor system. For example, the disclosed methods include pressurizing at least one pressure vessel in a bioreactor system at a pressure above atmospheric pressure and circulating heated fluid through the bioreactor system at a temperature above an atmospheric boiling point of the heated fluid.

Also, the disclosed implementations include methods, systems, and apparatuses for producing, storing, and/or providing superheated water within a closed bioreactor system. For example, one or more of the disclosed implementations include an apparatus configured to simultaneously produce process steam and superheated water. The disclosed systems also include various means for producing and/or storing superheated water for use during sterilization procedures.

Additional features and advantages of one or more implementations of the present disclosure are outlined in the description which follows, and in part will be obvious from the description, or may be learned by the practice of such example implementations.

### BRIEF DESCRIPTION OF THE DRAWINGS

Various implementations will be described and explained with additional specificity and detail through the use of the accompanying drawings, which are summarized below.

FIG. 1 illustrates an overview diagram of preparing a comestible cell-based food product in accordance with one or more implementations of the present disclosure.

FIG. 2 illustrates a system for cleaning-in-place (CIP) and sterilization-in-place (SIP) of process equipment within a closed bioreactor environment in accordance with one or more implementations of the present disclosure.

FIG. 3 illustrates a system for sterilization-in-place (SIP) of process equipment within a closed bioreactor environment utilizing direct-heated superheated water in accordance with one or more implementations of the present disclosure.

FIG. 4 illustrates a system for sterilization-in-place (SIP) of process equipment within a closed bioreactor environment utilizing indirect-heated superheated water in accordance with one or more implementations of the present disclosure.

FIG. 5 illustrates a system for cleaning-in-place (CIP) and sterilization-in-place (SIP) of process equipment within a closed bioreactor environment including a superheated water storage tank in accordance with one or more implementations of the present disclosure.

FIG. 6 illustrates a combination steam generator and superheated water generator in accordance with one or more implementations of the present disclosure.

FIG. 7 illustrates a system for cleaning-in-place (CIP) and sterilization-in-place (SIP) of process equipment within a closed bioreactor environment including a combination steam generator and superheated water generator in accordance with one or more implementations of the present disclosure.

FIG. 8 illustrates a bioreactor system and process flowchart for producing comestible cell-based food products, the system including a superheated water storage tank and a stratified energy storage tank in accordance with one or more implementations of the present disclosure.

FIG. 9 illustrates a flowchart of a series of acts for sterilizing a bioreactor system in accordance with one or more implementations of the present disclosure.

FIGS. 10A-10D illustrate an overview diagram of growing and processing different types of cells in accordance with one or more implementations of the present disclosure.

### DETAILED DESCRIPTION

This disclosure describes implementations of systems, apparatuses, and methods for generating and utilizing superheated water to sterilize process equipment in a closed bioreactor system. In one or more implementations, for example, the disclosed methods include sterilizing one or more vessels of a bioreactor system with fluid heated above an atmospheric boiling point of the fluid and pressurized above an atmospheric pressure to prevent evaporation of the fluid during a sterilization dwell time. The disclosed implementations also include methods, systems, and apparatuses for producing, storing, and/or providing superheated water within a closed bioreactor system configured to grow and process comestible cell-based food products.

To illustrate, in some implementations, the disclosed methods include pressurizing at least one pressure vessel in a bioreactor system at a pressure above atmospheric pressure and circulating heated fluid through the bioreactor system at a temperature above an atmospheric boiling point of the heated fluid (e.g., for a first period of time). Further, in some implementations, the disclosed methods include reducing a pressure within the at least one pressure vessel to cause the heated fluid therein to evaporate and further sterilize the at least one pressure vessel with the resulting depressurized steam (e.g., for a second period of time).

Further, in one or more implementations, the disclosed systems include one or more apparatuses for producing and/or storing superheated water in communication with a closed bioreactor system. For example, the disclosed systems can include one or more superheated water generators configured to heat pressurized fluid to a target temperature above an atmospheric boiling point of the fluid. Also, the disclosed systems can include one or more energy storage tanks configured to store and maintain process fluid at an elevated temperature and/or pressure for use in sterilizing process equipment of a bioreactor system. Moreover, in some implementations, the disclosed systems include a stratified energy storage tank configured to store process fluid at various temperature levels within a pressure vessel in communication with a closed bioreactor system.

The disclosed systems and methods provide several benefits relative to existing systems and methods for sterilizing process equipment of a closed bioreactor system. For example, existing systems commonly use steam for sterilization-in-place (SIP) procedures within the biopharmaceutical industry, as well as many related or analogous applications. Steam sterilization, however, can present various difficulties and disadvantages. For instance, culinary steam required for sterilization procedures is often costly and difficult to produce at the scale required for an industrial system, and energy loss during steam sterilization can further contribute to high costs and inefficiencies. Moreover, the high energy content of steam can react with surfaces and gases to cause corrosion of equipment when subjected thereto for significant durations, as is often the case in existing methods utilizing steam for sterilization-in-place (SIP) procedures. Further still, the high energy content of steam can present risks to personnel safety, thus requiring additional costs to prevent steam-related mishaps.

As mentioned, the disclosed systems and methods provide several benefits relative to existing systems and methods for sterilizing process equipment of a closed bioreactor system. In particular, the disclosed methods utilize superheated water (i.e., process fluid heated above an atmospheric boiling point and maintained in a liquid state by pressurization above an atmospheric pressure) for sterilization-in-place (SIP) of process equipment within a closed bioreactor system. By utilizing superheated water for sterilization-in-place (SIP) procedures, the disclosed methods reduce the total energy required for sterilization relative to existing methods that utilize culinary steam for sterilization-in-place (SIP).

Moreover, the disclosed systems implement improvements in process efficiency and reduced costs over existing systems by generating, storing, and utilizing superheated water for sterilization-in-place (SIP) procedures according to the various implementations described. For example, in some implementations, the disclosed systems include apparatuses for generating superheated water utilizing a variety of energy sources (e.g., direct heating or indirect heating using process steam). In one or more implementations, the discloses apparatuses for production of superheated water are less costly than existing apparatuses for producing culinary steam needed in existing SIP procedures.

Furthermore, in one or more implementations, the disclosed methods of utilizing superheated water reduce corrosion of process equipment during sterilization compared to existing methods. In certain implementations, the disclosed methods include reducing a pressure of superheated water to produce steam at a conclusion of sterilization-in-place (SIP) procedures (e.g., after circulation of superheated water for a dwell time). By utilizing steam in shorter intervals compared to existing methods, some implementations reduce corrosion inherent in steam sterilization.

As illustrated by the foregoing discussion, the present disclosure utilizes a variety of terms to describe features and advantages of the disclosed systems and methods. Additional detail is now provided regarding the meaning of such terms.

As used herein, the term “cells” refers to cells that are useful for forming a cell-based food product for consumption. Generally, cells may comprise non-human mesenchymal progeny. For instance, cells may comprise at least one of muscle cells, muscle progenitor cells, or muscle support cells. In particular, cells may comprise different cell types, such as one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, or other similar cell types. Furthermore, cells may comprise different types of progenitor cells, including myogenic progenitors, adipogenic progenitors, mesenchymal progenitors, or other types of progenitor cells. In some instances, the cells may comprise cells from distinct lineages, such as ectoderm or endoderm lineages, that have been transdifferentiated into cells useful for forming a cell-based food product for consumption, such as those cell types described above.

Relatedly, as used herein, the terms “cell culture media” or “culture media” refer to a liquid or gel comprising compounds that support the growth of cells. In particular, cell culture media comprises sources of energy and compounds to regulate the cell cycle. For example, a cell culture media can contain amino acids, vitamins, inorganic salts, glucose, dissolved gases, serum, growth factors, hormones, and attachment factors. The cell media may also help maintain pH and osmolarity during cell growth and proliferation.

As used herein, the term “cell mass” refers to a tissue or mass of cells. Cell mass refers to cells of cultivated meat gathered into a collective mass. In some implementations, the cell mass is comestible. Additionally, a cell mass can include grown cells that have been nourished by a growth medium to grow during a formation period within a cultivator. In some examples, a cell mass is grown from cells attached to a substrate in an adherent reactor.

As used herein, the terms “cell-based food product” and “cell-based animal food product” refer to a food product comprising non-human animal cells grown in vitro. For instance, the cell-based food product can include isolated cells from animals combined with other ingredients or additives such as, but not limited to, plant proteins, salts, flavorings, acids. Such products are interchangeably referred to as in vitro meat product, in vitro food product, lab grown meat, cultured food, or slaughter-free meat, depending on context.

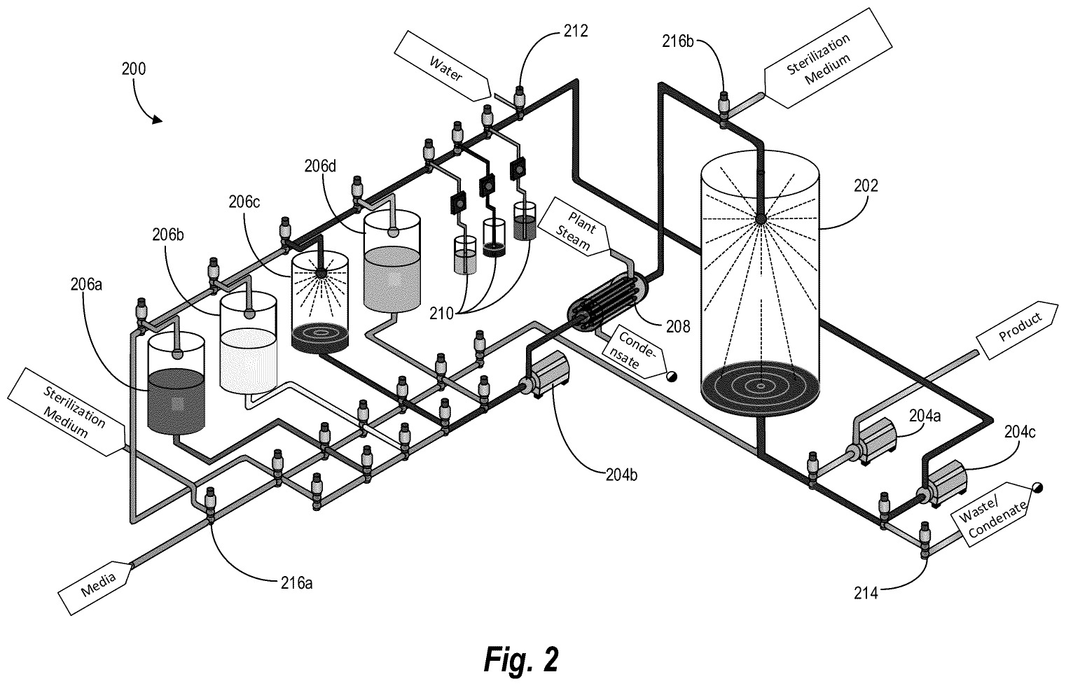

As used herein, the term “bioreactor” (or “cultivator”) refers to an apparatus in which cells are introduced, cultivated, and grown to form comestible meat cells of a cell-based food product. In particular, a bioreactor or cultivator refers to an environment that can hold cells and cell culture media. For example, a bioreactor system can comprise a sterile environment (e.g., devoid of foreign contaminants therein and separated/sealed from potential contaminants outside of a system boundary) having one or more bioreactor vessels (sometimes referred to as “suspension bioreactors” or “adherent bioreactors”) in fluidic communication with various sterile material supplies and sterile support equipment, such as described below in relation to FIGS. 2-8. For instance, an adherent bioreactor or cultivator can include an adherent reactor system that contains a substrate for growing adherent cells. Moreover, a suspension bioreactor or cultivator can include a substrate-free bioreactor system for growing cells in suspension.

As used herein, the term “suspension culture” (or “suspension”) refers to cells growing in an at least partially liquid growth medium in which cells grow, multiply, and/or maintain nourishment. In particular, a suspension includes an agitated growth medium that is housed in a container in which single cells or small aggregates of cells grow, multiply, and/or maintain nourishment from the nutrients of the agitated growth medium. Cells grown in suspension are not attached to a substrate and therefore differ from a conventional adherent culture.

As used herein, the terms “sterile environment,” “closed environment,” and “closed bioreactor system” refer to a space with regulated environmental factors and physical boundaries. In particular, a closed environment or system includes a space in which certain parameters, such as pressure, temperature, segregation, and exposure to outside elements, are controlled. More specifically, external contaminants are limited, or nonexistent, within a closed environment or system. For instance, a closed environment or system is isolated from contaminants found in air (e.g., ambient air). Further, a closed environment or system comprises an area that is contained on all sides. In particular, a closed bioreactor system comprises an enclosed environment including one or more fluidly connected spaces that are at least substantially closed off from external contaminants. In another example, a closed environment or system comprises a transfer mechanism, such as a series of sterile containers including a cultivator and a harvest collector. The series of containers may be sterilized and sealed prior to receiving cells. In another example, a closed environment or system comprises a series of pipes for transporting and processing cells. In yet another example, a closed environment or system includes means for preventing contamination from an external environment during the introduction or removal of materials and/or supporting equipment from within the boundaries of the closed environment or system.

As used herein, the term “harvest” or “harvesting” refers to a process of removing cells from a closed environment, such as a closed bioreactor system. For example, harvesting refers to the process of removing cells from a closed environment and exposing them to air. Harvesting may comprise removing adherent cells or suspension cells from a closed environment. In one or more implementations, harvesting comprises opening a harvest collector holding cells, wherein the cells are exposed to air (e.g., ambient air, filtered air, and/or refrigerated air) and any contaminants therein.

More specifically, harvesting cells can occur, in one or more implementations, when the cells are removed from the cultivator (e.g., bioreactor). In particular, harvesting occurs when removing the cells from the cultivator involves removing the cells from a closed environment or exposing the cells to air. Alternatively, the cells are removed from the cultivator (e.g., bioreactor) without removing the cells from the closed environment such that harvesting takes place after the cells are removed from the cultivator.

Along similar lines, harvesting cells can occur, in one or more implementations, when the cells are separated from a cell culture media (e.g., growth or nutrient media). In particular, when separating the cells from the cell culture media involves removing the cells from a closed environment or exposing the cells to air, harvesting occurs. Alternatively, the cells are separated from a cell culture media before removing the cells from the closed environment such that harvesting takes place after the cells are separated from a cell culture media.

As used herein, the term “process equipment” refers to components of a bioreactor system configured for processing (e.g., seeding, cultivating, and harvesting) comestible cell-based food products. In one or more implementations, for example, processing equipment includes any sterilizable component, such as but not limited to piping, valves, pumps, and tanks. Relatedly, as used herein, the term “pressure vessel” refers to a container designed to hold material at an elevated pressure (e.g., relative to an ambient or atmospheric pressure). For example, a pressure vessel can include a bioreactor, cultivator, energy storage tank, or other tanks included in a bioreactor system, such as tanks storing cell culture media, cleaning chemicals, mineral buffers, treated fluids, and so forth. Further, as used herein the term “energy storage tank” refers to an insulated reservoir for storing fluid (e.g., water) at a sustained temperature.

As used herein, “process steam,” also known as “plant steam,” refers to steam which is used in process applications such as a source of energy for process heating, process cooling, pressure control, and so forth. Relatedly, as used herein, the terms “clean steam” and “culinary steam,” used interchangeably, refer to sterile, safe steam that can be used to sterilize and clean items requiring a strict cleanliness criteria, such as components of a closed bioreactor system (e.g., process equipment as described above). In most cases, clean steam is produced by pumping a treated feedwater (e.g., purified, softened, and/or degassed water) through an evaporator, which when heated by a heat source (e.g., “indirectly” by process steam or “directly” by a fired boiler), evaporates to produce a pure steam for use in sterilization.

As used herein, the term “superheated water” refers to a fluid, such as but not limited to water, that is heated to a temperature above an atmospheric boiling point for the fluid and pressurized at a pressure above an atmospheric pressure to prevent evaporation of the fluid. In some implementations, for example, superheated water comprises a fluid treated with a mineral buffer to reduce corrosion of internal surfaces during sterilization of process equipment. In one or more implementations, superheated water is heated to a sterilization temperature between 120 and 140 degrees Celsius and pressurized to between 2 and 4 bars. Various other temperatures and pressures can be implemented to produce superheated water, as long as the pressure is sufficient to prevent boiling of the heated fluid at the corresponding fluid temperature. For example, in some implementations, the superheated water is heated to a temperature between 110 and 180 degrees Celsius.

As used herein, the term “cleaning-in-place” (CIP), sometimes referred to as “clean-in-place,” refers to methods of cleaning the interior surfaces of process equipment in a closed bioreactor system while maintaining a closed environment and without taking apart or otherwise disassembling the system during cleaning. For example, cleaning can include circulating a cleaning solution through pipes, vessels, filters, valves, and other equipment of a closed bioreactor system in a turbulent flow and/or via spray balls to remove particulates from within the various process equipment. Relatedly, as used herein, the term “sterilization-in-place” (SIP) refers to methods of sterilizing process equipment of a closed bioreactor system while maintaining a closed environment and without taking apart or otherwise disassembling the system during sterilization procedures. For example, sterilization can include removing, killing, and/or deactivating living organisms within a closed bioreactor system. Existing systems, for example, generally utilize superheated steam for a predetermined dwell time to achieve sterilization, whereas the methods disclosed herein utilize superheated water and/or other methods.

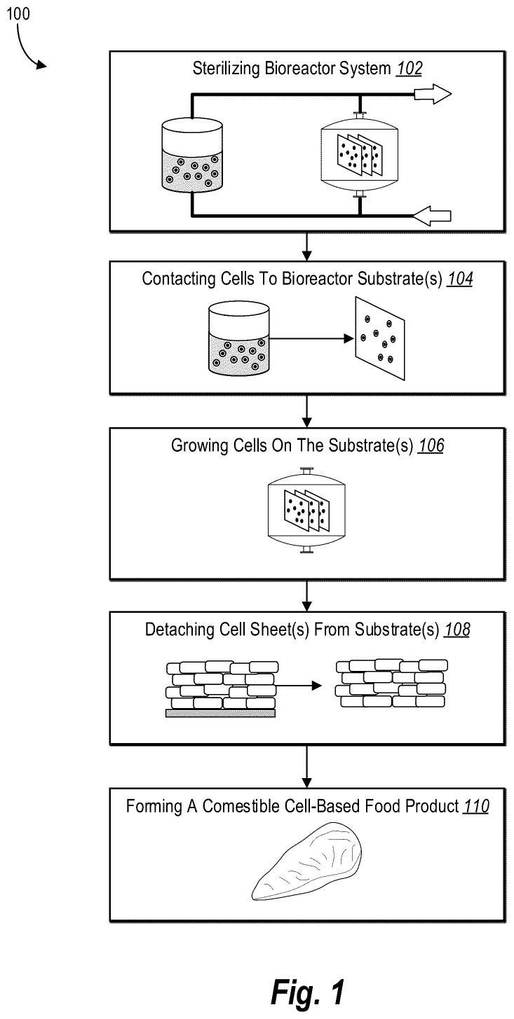

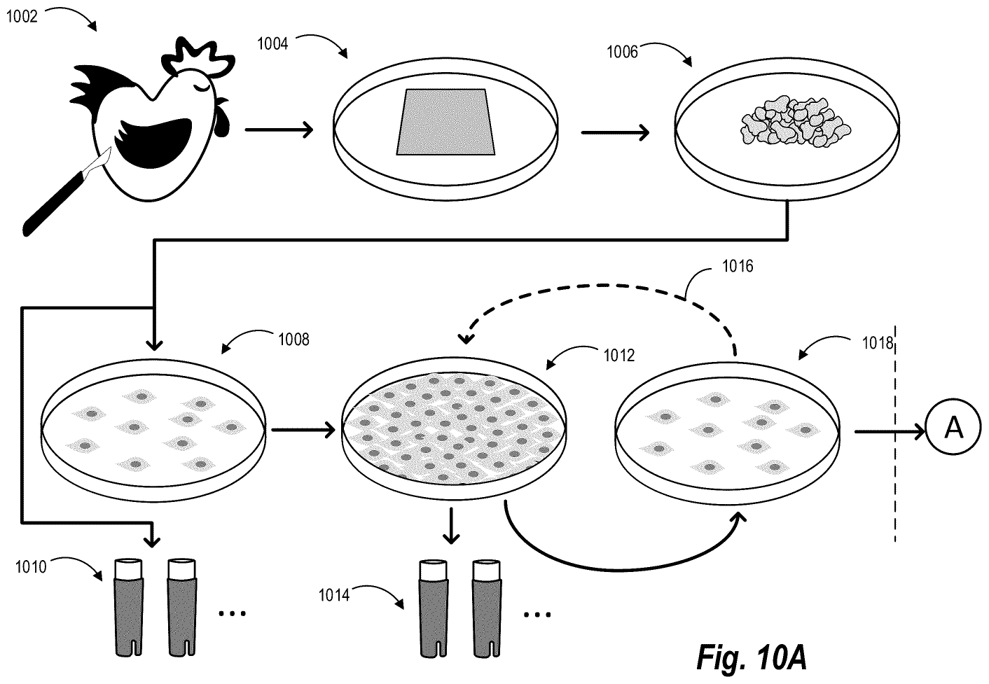

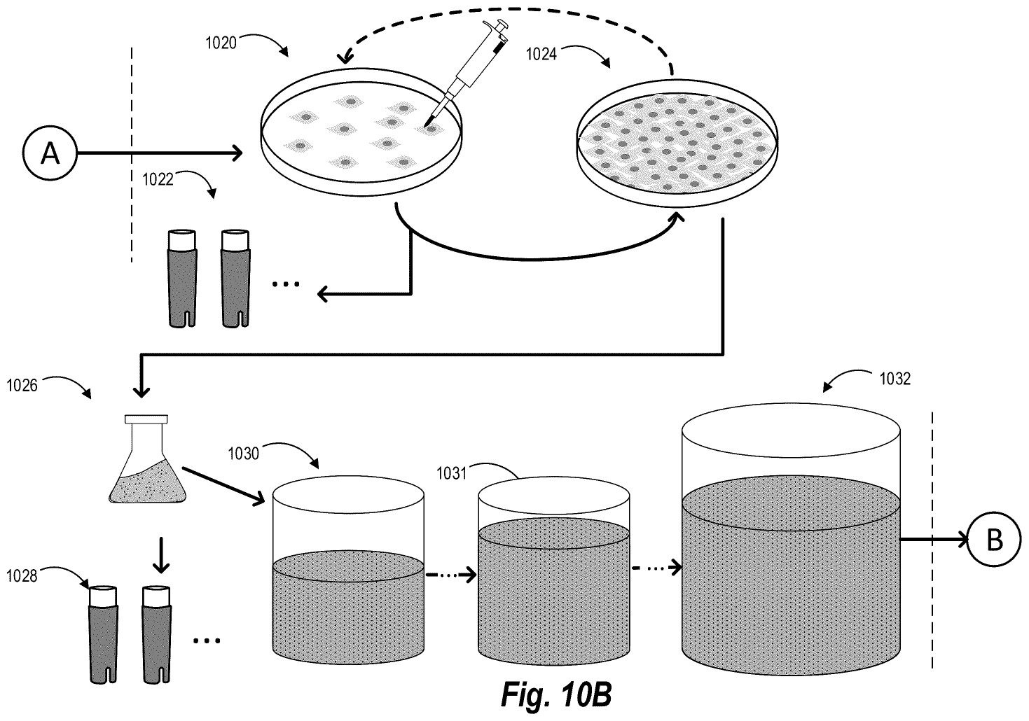

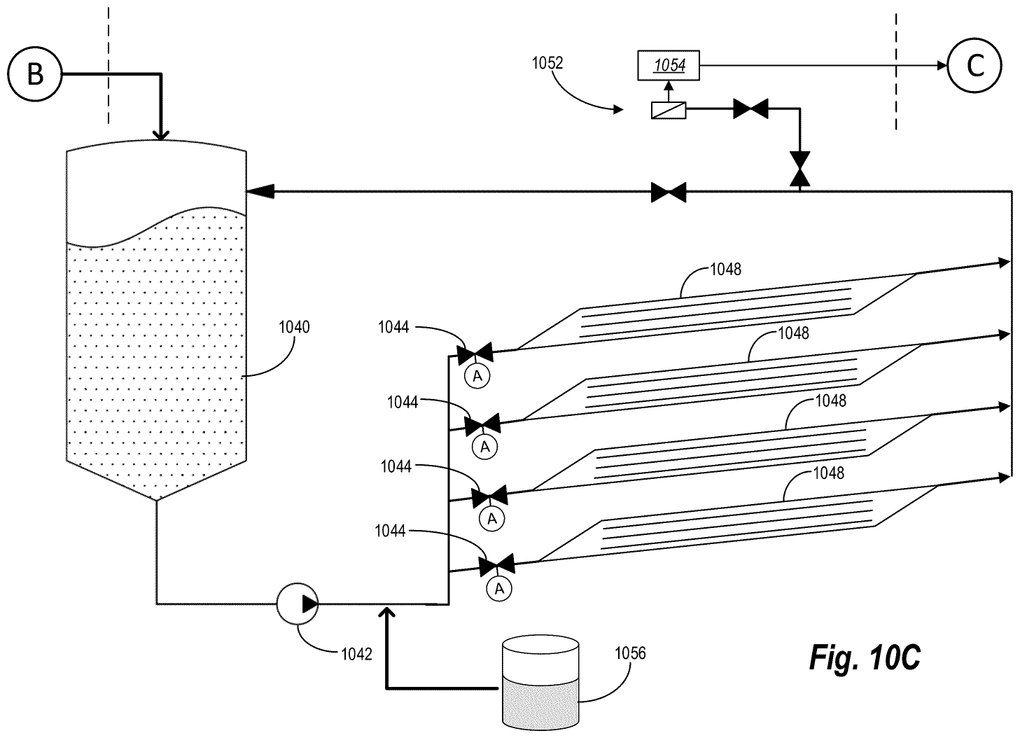

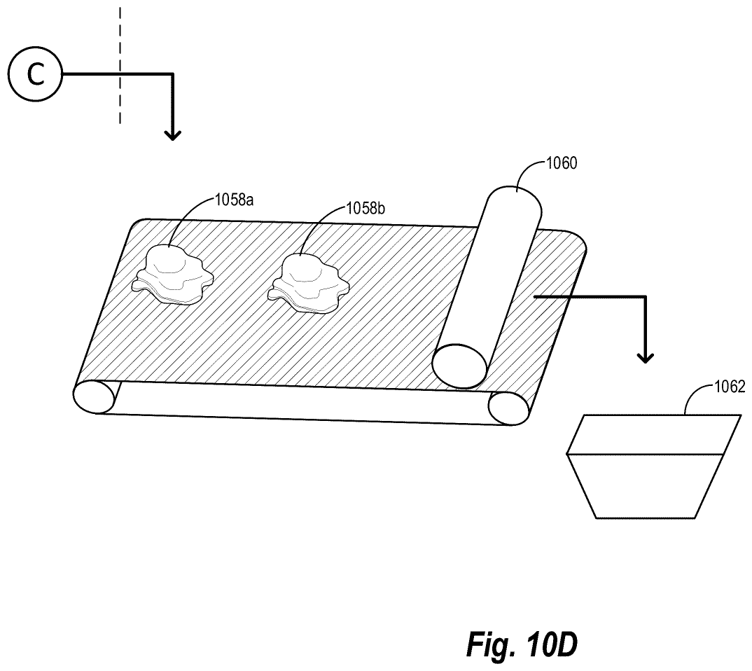

Additional detail will now be provided regarding the disclosed systems, apparatuses, and methods in relation to illustrative figures portraying example implementations. FIG. 1 illustrates an example process for preparing comestible cell-based food products in accordance with one or more implementations. By way of overview, FIG. 1 illustrates a series of acts 100 comprising an act 102 of sterilizing a bioreactor system, an act 104 of contacting cells to bioreactor substrate(s), an act 106 of growing cells on the substrate(s), an act 108 of detaching cell sheet(s) from substrate(s), and an act 110 of forming the cell sheet(s) into a comestible cell-based food product. FIGS. 10A-10D and the corresponding paragraphs herein further detail cultivating cells to form comestible cell-based food products in accordance with one or more implementations.

As shown in FIG. 1, in one or more implementations, a bioreactor system is prepared for initiating a process of growing cells therein by performing the act 102 of sterilizing the bioreactor system. As mentioned above, existing methods generally include sterilizing of bioreactor systems using steam, in particular culinary steam, whereas implementations described herein include sterilization by superheated water. In some implementations, steam is also utilized to complete the sterilization process by, for example, reducing a system pressure during sterilization by superheated water to cause the superheated water to evaporate within the bioreactor system.

As further shown in FIG. 1, in one or more implementations, meat cells (i.e., adherent cells) are prepared by performing the act 104 of contacting cells to one or more cell culture substrates of a bioreactor or bioreactor system. In one or more implementations, the cell culture substrates comprise a permeable substrate (e.g., permeable to physiological solutions) or an impermeable substrate (e.g., impermeable to physiological solutions). The substrate for the adherent cells can be flat, concave, or convex. Additionally, the substrate for the adherent cells can be textured so as to promote cell growth.

To illustrate, in one or more implementations, the adherent cells are prepared by contacting cell culture substrates to a cultivation infrastructure, including within a pipe-based bioreactor, a tank-based bioreactor, and so forth. In some implementations, the culturing of adherent cells can induce the production of extracellular matrix (ECM). Indeed, in one or more implementations, the ECM can act as an autologous scaffold to direct three-dimensional cellular growth. For example, in some implementations, the ECM can direct attachment, proliferation, and hypertrophy of cells on a plane perpendicular to the substrate. In addition, or in the alternative, in some implementations, the cultivation infrastructure may not comprise an exogenously added scaffold to promote self-assembly of a three-dimensional cellular biomass. In some implementations, the cultivation infrastructure may not comprise exogenous scaffolds such as a hydrogel or soft agar.

As mentioned above, adherent cells can be grown to form a cell sheet. Accordingly, an exemplary method of producing a comestible cell-based food product comprises: (a) providing fibroblasts and/or myoblasts from a non-human organism; (b) culturing the fibroblasts and/or myoblasts in media under conditions under which the fibroblasts and/or myoblasts grow in either suspension culture or adherent culture, wherein the media is substantially free of serum and other components derived from an animal.

Additionally, as shown in FIG. 1, in one or more implementations, the adherent cells are prepared by performing an act 106 of growing cells on the substrate(s). In one or more implementations, the adherent cells are grown on a suitable substrate that is specifically treated to allow cell adhesion and spreading, such as a surface located within a sterile enclosure of a bioreactor. The culturing conditions for the generation of the animal cells for a comestible cell-based food product are generally aseptic and sterile. For example, cells are injected into a cultivation tank, such as an adherent bioreactor. The cultivator (i.e., bioreactor) contains the substrate(s) (e.g., metal planks, sheets, or a lattice) to which cells can adhere. The cells are flowed through the enclosure of the bioreactor to allow cells to adhere to the substrate over time.

Before seeding the cells onto the substrate, in some implementations, the disclosed methods include preparing the substrate, such as by adding or flowing over adherent media to increase cell adherence to the substrate. As suggested above, in some implementations, the substrate is located within a bioreactor enclosure that is a sterile environment. To prepare the substrate in a bioreactor, the disclosed methods can include adding adherent media. The adherent media can be low in calcium to limit cellular clumping, so the cells spread out evenly across the substrate. The adherent media further facilitates attachment by the cells to the substrate. In some implementations, preparing the substrate further includes adding conditioning media and bringing the conditioning media up to temperature. The conditioning media further prepares the substrate by controlling pH, carbon dioxide, and oxygen levels within the cultivation tank. Additionally, the conditioning media may coat the substrates such that the adherence capability of the later seeded cells is enhanced.

The disclosed methods include growing the cells into a cellular tissue. Generally, the seeded cells (including the seeded initial cells and the previously unlanded cells) are grown in conditions that allow the formation of cellular tissue for a formation period. In some cases, the formation period can equal 4-14 days or other periods. During the formation period, cells may be provided with additional nutrients, media, growth factors, and other supplements to promote cellular growth. For example, the disclosed methods can include providing growth media. The growth media can include growth factors and beneficial proteins. At nutrient intervals during the formation period, additional feeds, amino acids, proteins, vitamins, minerals, and growth factors may be added to the cultivation tank to support growth in the seeded cells. Additionally, or alternatively, the disclosed methods include adding pre-harvest media before harvest. For instance, three days before harvest, a pre-harvest media including yeast concentrate may be added to the cultivation tank.

In one or more implementations, the adherent cells include cellular tissue of cultured meat gathered into a collective or agglomerated mass, including via growth on a substrate. In one or more implementations, the cultivation infrastructure for cultivating the adherent cells has a three-dimensional structure or shape for cultivating a monolayer of adherent cells. Additionally, in some implementations, the cultivation infrastructure can promote the adherent cells to form a three-dimensional growth of metazoan cells in the cultivation infrastructure to provide a scaffold-less self-assembly of a three-dimensional cellular biomass.

In some implementations, the adherent cells are grown on a three-dimensional cultivation infrastructure. The three-dimensional cultivation infrastructure may be sculpted into different sizes, shapes, and forms, as desired, to provide the shape and form for the adherent cells to grow and resemble different types of muscle tissues such as steak, tenderloin, shank, chicken breast, drumstick, lamb chops, fish filet, lobster tail, etc. The three-dimensional cultivation infrastructure may be made from natural or synthetic biomaterials that are non-toxic so that they may not be harmful if ingested. Natural biomaterials may include, for example, collagen, fibronectin, laminin, or other extracellular matrices. Synthetic biomaterials may include, for example, hydroxyapatite, alginate, polyglycolic acid, polylactic acid, or their copolymers. The three-dimensional cultivation infrastructure may be formed as a solid or semisolid support.

A cultivation infrastructure can be of any scale and support any volume of cellular biomass and culturing reagents. In some implementations, the cultivation infrastructure ranges from about 10 μL to about 100,000 L. In exemplary implementations, the cultivation infrastructure is about 10 μL, about 100 μL, about 1 mL, about 10 mL, about 100 mL, about 1 L, about 10 L, about 100 L, about 1000 L, about 10,000 L, or even about 100,000 L.

In one or more implementations, the comestible cell-based food product, unless otherwise manipulated to include, does not include vascular tissues, such as veins and arteries, whereas conventional meat does contain such vasculature, and contains the blood found in the vasculature. Accordingly, in some implementations, the comestible cell-based food product does not comprise any vasculature.

Likewise, comestible cell-based food product, although composed of muscle or muscle-like tissues, unless otherwise manipulated to include, does not comprise functioning muscle tissue. Accordingly, in some implementations, the cell-based meat does not comprise functioning muscle tissue. It is noted that features such as vasculature and functional muscle tissue can be further engineered into the cell-based meat, should there be a desire to do so.

Also, as shown in FIG. 1, the adherent cells can be prepared by performing an act 108 of detaching a cell sheet from the substrate. In one or more implementations, the adherent cells are harvested by detaching a cell sheet from the substrate. In particular, the adherent cells are harvested based on various factors. The adherent tissue may be harvested after a proliferation time period. For example, the adherent cells are harvested after the cells have been growing for anywhere between 4 and 14 days. In another example, the adherent cells are harvested based on completing a proliferation phase. More specifically, the adherent cells may be harvested when the cell sheet starts contracting and stops growing. For instance, the cell sheet may begin to detach from the substrate. In one or more implementations, the cell sheet is detached from the substrate after adding pre-harvest media before harvest.

The method of FIG. 1 can optionally involve reducing the moisture and/or size of an agglomeration (e.g., the cell sheet) of adherent cells. For example, the act 110 can involve reducing a moisture content in the adherent cells by vacuum drying the adherent cells. In addition, in some implementations, the act 110 involves reducing a size of the meat cells via chopping, or other technique. Furthermore, the act 110 can involve forming the cells into the shape of a meat product (e.g., chicken breast, steak).

Furthermore, while FIG. 1 illustrates an example process of growing adherent cells within a sterile bioreactor system comprising at least one adherent bioreactor, one or more implementations include growth of cells in suspension within a sterile bioreactor system comprising at least one suspension bioreactor vessel. Indeed, one should appreciate that the systems and methods disclosed herein are generally applicable to bioreactor systems configured for growing adherent cells on one or more substrates or growing suspension cells within a suspension bioreactor vessel.

To further illustrate, FIG. 2 shows a system for cleaning-in-place (CIP) and sterilization-in-place (SIP) of process equipment within a closed bioreactor environment. In particular, FIG. 2 illustrates a system 200 comprising equipment and components for cleaning and sterilizing a bioreactor 202 within a closed environment. As illustrated, for example, the bioreactor 202 can be emptied upon completion of a production process (e.g., by removing product therefrom using a harvesting pump 204a), then cleaned and sterilized prior to subsequent operations for producing comestible cell-based food products.

As shown in FIG. 2, the system 200 includes a first reservoir 206a containing a caustic solution for breaking down organic compounds within the bioreactor 202. In one or more embodiments, the caustic solution comprises an alkali. For example, in one or more implementations, the caustic solution comprises sodium hydroxide. The caustic solution can be circulated into the bioreactor 202 with a CIP system pump 204b to initiate cleaning-in-place (CIP) procedures. The caustic solution, for example, can be pumped through system pipes at relatively high cleaning velocities (e.g., 1.5-3.0 m/s) and sprayed with turbulence into the bioreactor 202 (e.g., via a spray ball) to disrupt, remove, and/or dissolve organic compounds (and other materials) from inner surfaces of the pipes and the bioreactor 202. In some implementations, the caustic solution can be heated for circulation by a heat exchanger 208 (e.g., using plant steam as a heat source) integrated with the system 200. Also, the system 200 includes supplemental reservoirs 210, one of which can provide additional caustic solution to maintain caustic concentration during circulation thereof. In some implementations, the caustic solution is returned to the first reservoir 206a after a predetermined dwell time. Alternatively, a portion or all of the caustic solution can be discarded from the system 200 via a waste valve 214.

As also shown in FIG. 2, the system 200 includes a second reservoir 206b containing a rinse fluid, such as a purified and/or otherwise treated water. Upon draining of the caustic solution from the bioreactor 202, for instance, the rinse fluid can be circulated through the system 200, via the CIP pump 204b, to perform an intermediate rinse to remove residues and materials that remain after the aforementioned caustic wash. Further, in some implementations, the rinse fluid from the second reservoir 206b can be heated by the heat exchanger 208 during circulation into the system 200. During the intermediate rinse of the system 200, rinse fluid can be drained from the system via waste valve 214 or reclaimed for subsequent CIP processes by flowing at least a portion of the used fluid into a third reservoir 206c utilizing a return pump 204c.

Furthermore, as shown in FIG. 2, the system 200 includes a fourth reservoir 206d containing an acid solution for further caustic neutralization and removal of mineral deposits from the bioreactor 202 and other components of the system 200. For example, the acid solution can be circulated through system pipes and into the bioreactor 202, generally without heating, via the CIP pump 204b. As also shown, the acid solution can be supplemented to maintain acid concentration during the acid wash by one of the supplemental reservoirs 210. In one or more implementations, acid solution is recirculated into the fourth reservoir 206d for use in subsequent CIP processes. In one or more implementations the acid solution comprises peracetic acid, hydrogen peroxide, other acids, or combinations thereof.

Finally, at a conclusion of the acid wash, the system 200 can undergo a final rinse from the second reservoir 206b, from a water source via valve 212, or by a combination thereof. Rinse fluid from the final rinse can either be drained from the system 200 via waste valve 214 or at least partially reclaimed and stored in the third reservoir 206c for subsequent use in CIP procedures. For example, reclaimed rinse fluid from the third reservoir 206c can be circulated in the system 200 as a pre-rinse prior to circulation of the caustic solution from the first reservoir 206a, as described above. In some implementations, one of the supplementary reservoirs 210 also provides a sanitation solution for sanitizing the bioreactor 202 and other components of the system 200 (e.g., to at least partially disinfect the system 200) in preparation for sterilization-in-place (SIP).

Moreover, as shown in FIG. 2, the system 200 includes valves 216a and 216b for introducing a sterilization medium into the bioreactor 202 and other components of the system 200. In existing systems, for context, culinary steam is generally utilized during sterilization-in-place (SIP) procedures to implement complete sterilization of the target process equipment. As mentioned previously and as described in further detail below, implementations of the present disclosure include utilizing superheated water in place of culinary steam for sterilization-in-place of closed bioreactor systems, such as the system 200.

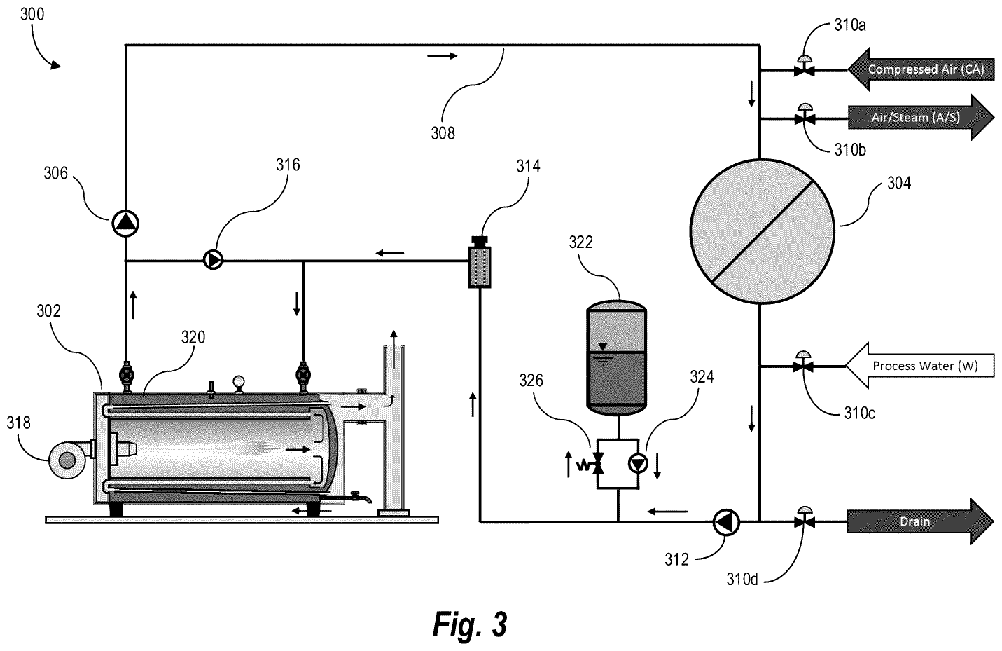

As mentioned previously, in one or more implementations, the disclosed systems and methods utilize superheated water for sterilization-in-place (SIP) of process equipment within a closed bioreactor environment. For example, FIG. 3 illustrates a system 300 comprising a superheated water generator 302 configured to directly heat fluid (e.g., treated water) to produce superheated water for sterilization-in-place (SIP) of a bioreactor 304 and/or other process equipment within a closed environment.

As shown in FIG. 3, the superheated water generator 302 comprises a direct-heated, flooded boiler having a burner 318 configured to heat fluid (e.g., process water provided via valve 310c) within a pressurized chamber 320 to reach a desired sterilization temperature. In some implementations, for example, the target sterilization temperature is between 120 and 140 degrees Celsius. To maintain the heated fluid in a liquid state (i.e., as superheated water), the fluid is pressurized within the superheated water generator 302 (and within the system 300) to a pressure sufficient to sustain the boiling point of the fluid at a temperature greater than the sterilization temperature to which the fluid is heated. In some implementations, for example, the pressure within the superheated water generator 302 (and within the system 300) is maintained between 2 and 4 bars. In some implementations, increased fluid temperatures and corresponding pressures are implemented, depending upon predetermined requirements for the system undergoing sterilization.

Also, as shown in FIG. 3, the system 300 is configured to circulate (e.g., via circulation pumps 306, 312) superheated water produced by the superheated water generator 302 into the bioreactor 304 (and other process equipment) through pipe 308 for sterilization-in-place (SIP) thereof. As previously mentioned, for example, in one or more implementations, superheated water is circulated within the bioreactor 304 for a predetermined dwell time. As illustrated, the system 300 includes a strainer 314 for filtration of the fluid prior to entry into the superheated water generator 302. Also, excess water, contaminated water, or otherwise unwanted fluids can be drained from the system 300 via a drainage valve 310d.

During sterilization-in-place (SIP), the fluid temperature can be maintained by introducing additional fluid and/or by re-heating fluid with the superheated water generator 302, with further temperature regularization provided by a return pump 316. Moreover, pressurization of the system 300 is implemented by a backpressure of compressed air provided via an input valve 310a and can be adjusted/evacuated via an output valve 310b. In addition, the system 300 includes a pressure hold tank 322 with a corresponding pressure regulating pump 324 and an overflow valve 326 for regularizing fluid pressure within the system 300.

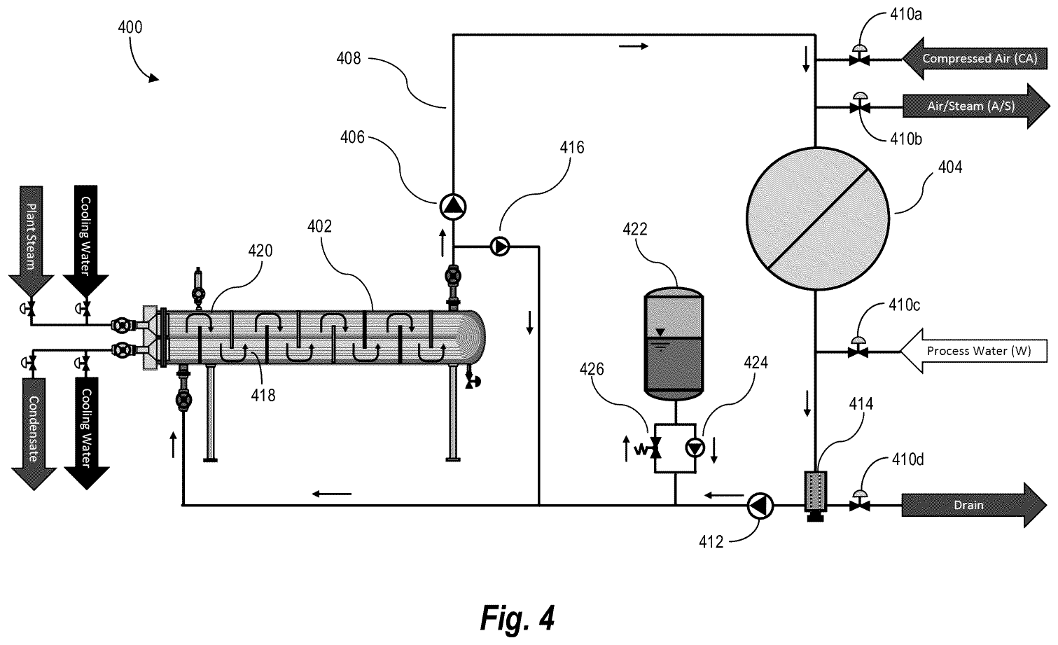

As mentioned, implementations of the disclosed systems and methods can utilize process steam to generate superheated water for sterilization-in-place (SIP) of a closed bioreactor system. For example, FIG. 4 illustrates a system 400 comprising a superheated water generator 402 configured to utilize process steam to indirectly heat fluid (e.g., treated water) to produce superheated water for sterilization-in-place (SIP) of a bioreactor 404 and/or other process equipment within a closed environment.

As shown in FIG. 4, the superheated water generator 402 comprises an indirect-heated, flooded boiler having a steam enclosure 418 configured to transfer heat to a pressurized chamber 420 containing fluid (e.g., process water provided via valve 410c) to reach a desired sterilization temperature. As mentioned above, in some implementations, for example, the target sterilization temperature is between 120 and 140 degrees Celsius. To maintain the heated fluid in a liquid state (i.e., as superheated water), the fluid is pressurized within the superheated water generator 402 (and within the system 400) to a pressure sufficient to sustain the boiling point of the fluid at a temperature greater than the sterilization temperature to which the fluid is heated. In some implementations, for example, the pressure within the superheated water generator 402 (and within the system 400) is maintained between 2 and 4 bars. In some implementations, increased fluid temperatures and corresponding pressures are implemented, depending upon predetermined requirements for the system undergoing sterilization.

Also, as shown in FIG. 4, the system 400 is configured to circulate (e.g., via circulation pumps 406, 412) superheated water produced by the superheated water generator 402 into the bioreactor 404 (and other process equipment) and through pipe 408 for sterilization-in-place (SIP) thereof. As previously mentioned, for example, in one or more implementations, superheated water is circulated within the bioreactor 404 for a predetermined dwell time. As illustrated, the system 400 includes a strainer 414 for filtration of the fluid prior to entry into the superheated water generator 402. Also, excess water, contaminated water, or otherwise unwanted fluids can be drained from the system 400 via a drainage valve 410d.

During sterilization-in-place (SIP), the fluid temperature can be maintained by introducing additional fluid and/or by re-heating fluid with the superheated water generator 402, with further temperature regularization provided by a return pump 416. Moreover, pressurization of the system 400 is implemented by a backpressure of compressed air provided via an input valve 410a and can be adjusted/evacuated via an output valve 410b. In addition, the system 400 includes a pressure hold tank 422 with a corresponding pressure regulating pump 424 and an overflow valve 426 for regularizing fluid pressure within the system 400.

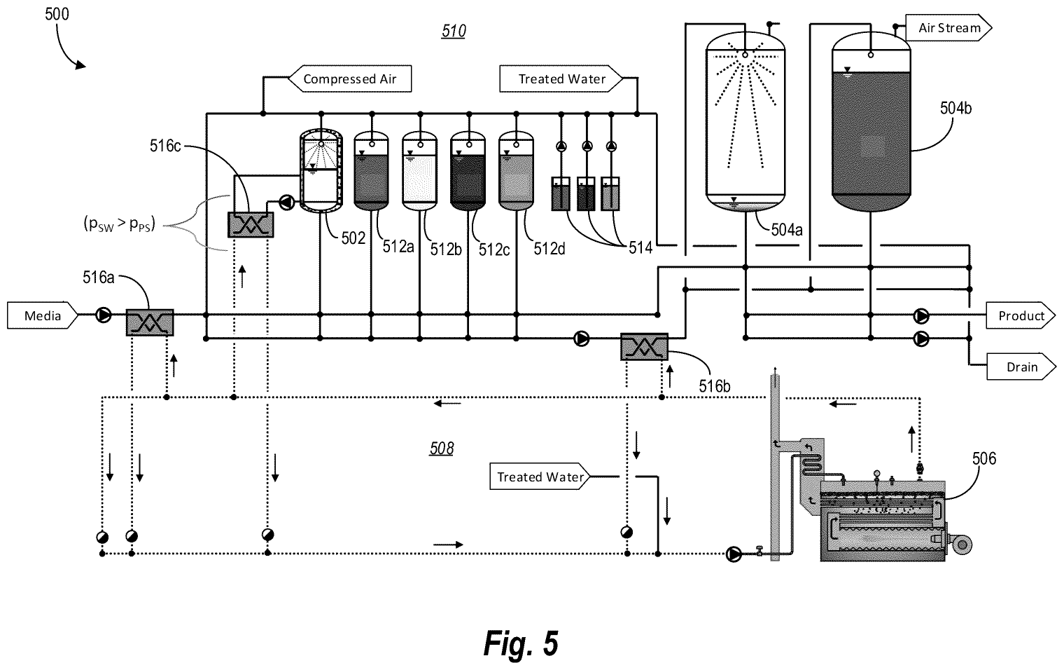

Furthermore, in some implementations, a source of superheated water is integrated within a closed bioreactor system including equipment for cleaning-in-place (CIP) and sterilization-in-place (SIP) procedures. For example, FIG. 5 illustrates a system 500 comprising an integrated CIP/SIP system 510 within a closed environment. In particular, FIG. 5 illustrates the system 500 having a superheated water (SHW) storage tank 502 containing superheated water for sterilization-in-place (SIP) of bioreactors 504a, 504b and other process equipment.

As shown in FIG. 5, the CIP/SIP system 510 includes a SHW storage tank 502 configured to store superheated water at a selected temperature and pressure. In some implementations, for example the SHW storage tank 502 comprises an insulated pressure vessel for storing fluid (e.g., treated water) at a pressure above an atmospheric pressure and a temperature above an atmospheric boiling point of the stored fluid. As also illustrated, the system 500 generates superheated water for storage within the SHW storage tank 502 by utilizing process steam provided by a process steam network 508 to heat treated water. The treated water used for superheated water can include pretreated water that, for example, is filtered, reduced in mineral content or otherwise modified in chemical composition, adjusted in ion content, and/or supplemented with complexing agents to prevent fouling on heat exchangers and deposit formation within process equipment during sterilization. Moreover, in some implementations, an alkalinity (i.e., pH level) of the treated water is adjusted to prevent corrosion in process equipment subjected thereto, such as by adding alkali and/or buffers to achieve a neutral pH value of 7 or a slightly alkaline pH value greater than 7.

In some implementations, for example, the superheated water can be produced by heat transfer from process steam via a steam-heated generator, such as but not limited to the superheated water generator 402 shown in FIG. 4 and described in further detail above. Alternatively, in some implementations, the superheated water can be produced by a direct-heated generator, such as but not limited to the superheated water generator 302 shown in FIG. 3 and described in further detail above.

As also shown in FIG. 5, the system 500 includes the process steam network 508 for generating and utilizing process steam in various procedures within the closed environment of the system 500. For instance, the process steam network 508 includes a process steam generator 506 configured to generate process steam for use in heating, for example, cell culture media via a heat exchanger 516a, caustic solution and/or rinse fluid via a heat exchanger 516b, and/or superheated water via the superheated water generator 516c. As indicated in FIG. 5, the process steam utilized to generate the superheated water can have a lower pressure (Pps) compared to the superheated water pressure (psw) so that the superheated water is maintained in a liquid state.

As shown in FIG. 5, the CIP/SIP system 510 also includes multiple reservoirs 512a-512d in communication with the system 500 for cleaning-in-place (CIP) of the bioreactors 504a-504b (and other process equipment). For instance, the reservoirs 512a-512d include a first reservoir 512a containing a caustic solution, a second reservoir 512b containing a rinse fluid (e.g., treated water), a third reservoir 512c containing reclaimed rinse fluid, and a fourth reservoir 512d containing an acid solution. Further, the CIP and SIP system 510 includes a series of supplementary reservoirs 514 for supplementing the solutions and fluids within the multiple reservoirs 512a-512d.

Accordingly, the foregoing features and components of the CIP/SIP system 510 of system 500 can be utilized to conduct cleaning-in-place and sterilization-in-place procedures. As shown in FIG. 5, for example, upon completion of a production cycle, cultivated cells can be removed from bioreactor 504a, and any excess fluids drained therefrom in preparation for cleaning-in-place (CIP) thereof. In some implementations, the second bioreactor 504b can be sealed off or otherwise not connected to the CIP/SIP system 510 during cleaning-in-place and sterilization-in-place of the first bioreactor 504a. Alternatively, in some implementations, both bioreactors 504a and 504b can be drained of product and simultaneously cleaned and sterilized according to the disclosed methods.

Following the cleaning-in-place (CIP) procedures (e.g., as described above in relation to FIG. 2), the bioreactor 504a can be prepared for sterilization-in-place (SIP) procedures. In some implementations, for example, the system 500 (including the bioreactor 504a and/or 504b to be sterilized) is pressurized by introducing compressed air (e.g., via a compressor) prior to circulating superheated water therein from the SHW storage tank 502, in order to prevent or reduce boiling of the superheated water as it enters the closed system. With the system pressure sufficiently raised within the process equipment to be sterilized, in some implementations, superheated water is then circulated from the SHW storage tank 502 by one or more pumps for a predetermined dwell time. In one or more embodiments, for example, the predetermined dwell time is at least 5 minutes.

In one or more implementations, for example, superheated water is circulated at one or more flow velocities between 0.5 and 6.0 m/s to promote heat transfer through turbulent flows and rapid, homogeneous heating to a sterilization temperature, as well as homogeneous dispersion by spray devices within the bioreactor(s) 504a, 504b being sterilized. In some implementations, the superheated water is circulated at one or more flow velocities between 1 and 3 m/s. Further, pressure losses can be compensated (e.g., by increasing system backpressure provided via compressed air) during the predetermined dwell time to maintain the superheated water in a liquid state. Also, temperature losses in the superheated water (e.g., due to energy transfer to the process equipment) can be compensated with additional superheated water from the SHW storage tank 502.

Furthermore, in some implementations, upon completion of the predetermined dwell time (i.e., when the fluid temperature has been maintained within the process equipment for a requisite dwell time), the pressure of the system 500 can be reduced to cause the superheated water to evaporate. In consequence of the evaporation, steam is generated within the process equipment locally to further sterilize the interior surfaces thereof. In some implementations, the pressure can be reduced intermittently during the dwell time to intermittently generate steam during the sterilization-in-place (SIP) procedure.

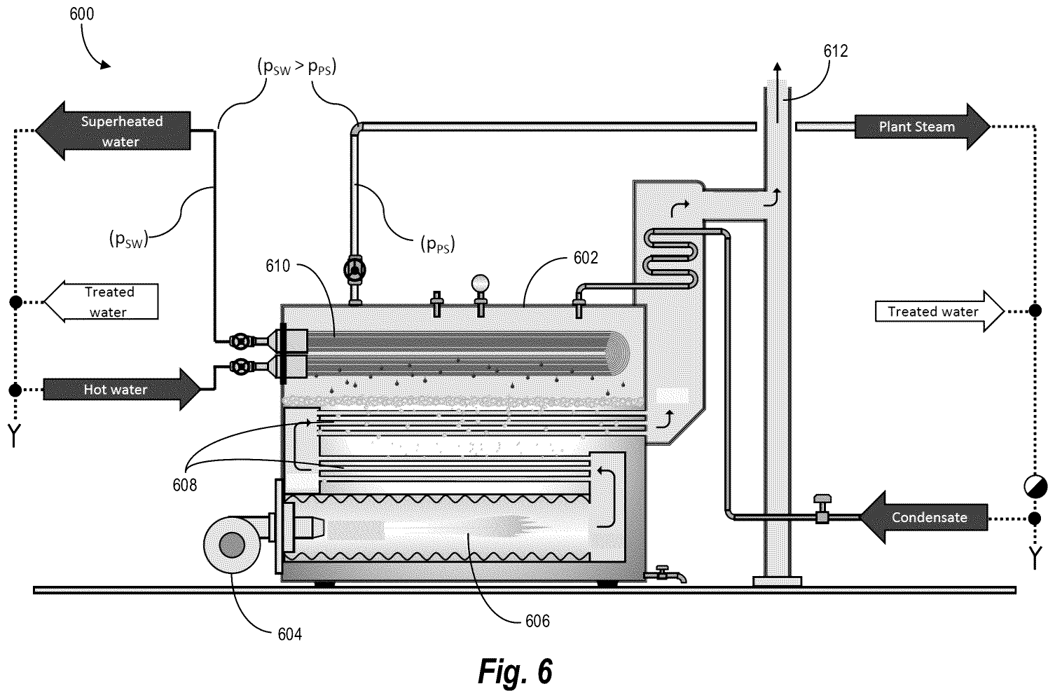

As previously mentioned, the disclosed systems can include an apparatus configured for combined generation of process steam and superheated water to be utilized in a closed bioreactor system. For example, FIG. 6 illustrates a combination steam generator and superheated water generator 600. In particular, the combination steam generator and superheated water generator 600 can simultaneously generate usable steam and superheated water with resulting reductions in energy loss.

As shown in FIG. 6, the combination steam generator and superheated water generator 600 includes an insulated enclosure 602 with a lower boiler section 606 heated directly by a burner 604. In addition, the lower boiler section 606 includes a lower heat exchanger 608 configured to produce steam by heating condensate delivered thereto. As shown, excess heat from the lower boiler section 606 is released from a chute 612. Further, a tubular heat exchanger 610 is also disposed within the insulated enclosure 602 and filled with treated water to be converted into superheated water. For instance, in some implementations, treated water is pressurized within the tubular heat exchanger to a pressure sufficient to prevent evaporation of the treated water as the temperature thereof is increased via the steam produced by the lower boiler section 606.

As process steam produced by the lower boiler section 606 rises and comes into contact with an outer surface of the tubular heat exchanger 610, heat transfers from the steam to the treated water within the tubular heat exchanger 610 and causes the encountered steam to condense into a liquid condensate. As illustrated, the resultant condensate falls from the tubular heat exchanger 610 onto the lower heat exchanger 608 of the lower boiler section 606, which in turn converts the condensate into additional process steam. In one or more implementations, the superheated water output from the tubular heat exchanger 610 has a greater pressure (Psw) than the pressure (Pps) of steam generated by the lower heat exchanger 608.

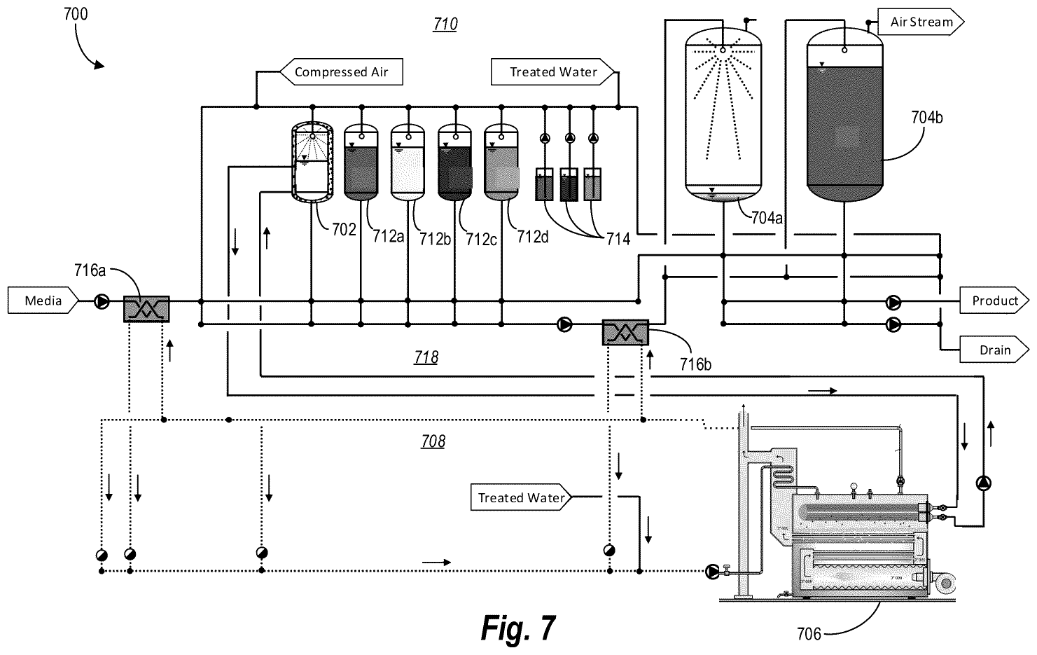

As also illustrated, the generated steam can be accessed separately from the generated superheated water. In some implementations, for example, a steam outlet valve is operable to release process steam into an integrated bioreactor system or CIP/SIP system. Further, in some implementations, the superheated water comprises sterile fluid conditioned for sterilization-in-place of a closed bioreactor system, whereas the process steam is indirectly implemented (e.g., by use in heat exchangers as discussed in relation to FIGS. 5 and 7) in various production processes (e.g., as described in relation to FIGS. 5 and 7). Alternatively, in one or more implementations, the combination steam generator and superheated water generator 600 is configured to produce clean steam (i.e., culinary steam) for utilization in sterilization procedures and other processes requiring clean steam.

Furthermore, in some implementations, a combination steam generator and superheated water generator is integrated within a closed bioreactor system including equipment for cleaning-in-place (CIP) and sterilization-in-place (SIP) procedures. For example, FIG. 7 illustrates a system 700 comprising an integrated CIP/SIP system 710 within a closed environment. In particular, FIG. 7 illustrates the system 700 having a superheated water (SHW) storage tank 702 storinging superheated water for sterilization-in-place (SIP) of bioreactors 704a, 704b and other process equipment. Moreover, the system 700 includes a combination superheated water and steam generator 706 for generating superheated water and process steam for utilization in various CIP and SIP procedures.

As shown in FIG. 7, the CIP/SIP system 710 includes the SHW storage tank 702 configured to store superheated water at a selected temperature and pressure. In some implementations, for example the SHW storage tank 702 comprises an insulated pressure vessel for storing fluid (e.g., treated water) at a pressure above an atmospheric pressure and a temperature above an atmospheric boiling point of the stored fluid. As also illustrated, the system 700 generates superheated water for storage within the SHW storage tank 702 (and subsequent use in sterilization of process equipment) by utilizing process steam as steam is generated within an insulated enclosure of the combination superheated water and steam generator 706 (e.g., as further described above in relation to FIG. 6). As shown, the superheated water can be delivered to the SHW storage tank 702 via a superheated water network 718. Alternatively, in some embodiments, superheated water can be provided directly to a bioreactor system during sterilization-in-place (SIP).

As mentioned, the treated water used for superheated water can include pretreated water that, for example, is filtered, reduced in mineral content, adjusted in ion content, and/or supplemented with complexing agents to prevent fouling on heat exchangers and deposit formation within process equipment during sterilization. Moreover, in some implementations, a pH level of the treated water is adjusted to prevent corrosion, such as by adding alkali and/or buffers to achieve a neutral pH value of 7 or a slightly alkaline pH value greater than 7.

As also shown in FIG. 7, the system 700 includes the process steam network 708 for utilizing process steam generated by the combination superheated water and steam generator 706 in various procedures within the closed environment of the system 700. For instance, the process steam network 708 includes heat exchangers for use in heating, for example, cell culture media via a heat exchanger 716a, as well as caustic solution and/or rinse fluid via a heat exchanger 716b.

As further shown in FIG. 7, the CIP/SIP system 710 also includes multiple reservoirs 712a-712d in communication with the system 500 for cleaning-in-place (CIP) of the bioreactors 704a-704b (and other process equipment). For instance, the reservoirs 712a-712d include a first reservoir 712a containing a caustic solution, a second reservoir 712b containing a rinse fluid (e.g., treated water), a third reservoir 712c containing reclaimed rinse fluid, and a fourth reservoir 712d containing an acid solution. Further, the CIP and SIP system 710 includes a series of supplementary reservoirs 714 for supplementing the solutions and fluids within the multiple reservoirs 712a-712d.

Accordingly, the foregoing features and components of the CIP/SIP system 710 of system 700 can be utilized to conduct cleaning-in-place and sterilization-in-place procedures. As shown in FIG. 7, for example, upon completion of a production cycle, cultivated cells can be removed from bioreactor 704a, and any excess fluids drained therefrom in preparation for cleaning-in-place (CIP) thereof. In some implementations, the second bioreactor 704b can be sealed off or otherwise not connected to the CIP/SIP system 710 during cleaning-in-place and sterilization-in-place of the first bioreactor 704a. Alternatively, in some implementations, both bioreactors 704a and 704b can be drained of product and simultaneously cleaned and sterilized according to the disclosed methods.

Following the cleaning-in-place (CIP) procedures (e.g., as described above in relation to FIG. 2), the bioreactor 704a can be prepared for sterilization-in-place (SIP) procedures. In some implementations, for example, the system 700 (including the bioreactor 704a and/or 704b to be sterilized) is pressurized by introducing compressed air or otherwise prior to circulating superheated water therein from the SHW storage tank 702, in order to prevent or reduce boiling of the superheated water as it enters the closed system. With the system pressure sufficiently raised within the process equipment to be sterilized, in some implementations, superheated water is then circulated from the SHW storage tank 702 by one or more pumps for a predetermined dwell time.

In one or more implementations, for example, superheated water is circulated at one or more flow velocities between 0.5 and 6.0 m/s to promote heat transfer through turbulent flows and rapid, homogeneous heating to a sterilization temperature, as well as homogeneous dispersion by spray devices within the bioreactor(s) 704a, 704b being sterilized. In some implementations, the superheated water is circulated at one or more flow velocities between 1 and 3 m/s. Further, pressure losses can be compensated during the predetermined dwell time to maintain the superheated water in a liquid state. Also, temperature losses in the superheated water (e.g., due to energy transfer to the process equipment) can be compensated with additional superheated water from the SHW storage tank 702.

Furthermore, in some implementations, upon completion of the predetermined dwell time (i.e., when the fluid temperature has been maintained within the process equipment for a requisite dwell time), the pressure of the system 700 can be reduced to cause the superheated water to evaporate. In consequence of the evaporation, steam is generated within the process equipment locally to further sterilize the interior surfaces thereof. In some implementations, the pressure can be reduced gradually, intermittently, or both during the dwell time to gradually and/or intermittently generate steam during the sterilization-in-place (SIP) procedure.

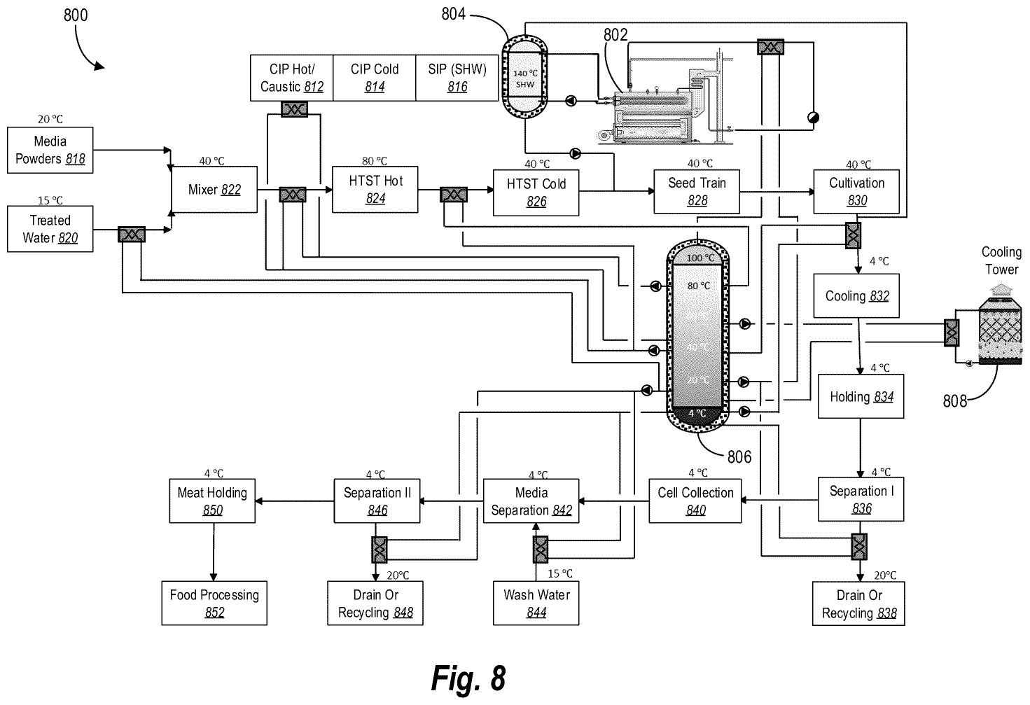

As previously mentioned, in one or more implementations, the disclosed system includes a stratified energy storage tank containing process fluid at various temperatures. For example, FIG. 8 illustrates a system 800 and overlaid process flowchart of methods for producing comestible cell-based meat products, the system including a stratified energy storage tank 806 storing process fluid (e.g., treated water) at a plurality of temperatures. As also illustrated, the system 800 further includes a combination superheated water and steam generator 802 and a superheated water (SHW) storage tank 804.

As shown in FIG. 8, the stratified energy storage tank 806 comprises an insulated enclosure with multiple layers of fluid maintained at different temperatures. In some implementations, for example, each layer is separated by a thermocline configured to prevent heat conduction between two or more stacked layers having temperature variability within the stratified energy storage tank 806. In general, hot water is stratified at the top and cold water at the bottom. This stratification is reinforced by flowing heated fluids through or around the top of the energy storage tank 806 to maintain its high temperature, while doing the same for colder fluids at the bottom of the energy storage tank 806. Stratification layers of the energy storage tank 806 can be further reinforced by drawing fluid from the central area of the thermocline and with low inflow and mixing speeds, while high inflow and mixing speeds can lead to homogenization and breakdown of the thermocline. Accordingly, process fluid at a plurality of temperatures can be provided by the stratified energy storage tank 806 for utilization in various steps of a process for producing comestible cell-based food products. Alternatively, in one or more implementations, various temperatures of process fluid are stored in individual energy storage tanks for each respective temperature. As also illustrated, in some implementations, heated fluid can be provided to the stratified energy storage tank 806 by the combined superheated water and steam generator 802. As also illustrated, relatively cold process fluid stored at a lower tier of the stratified energy storage tank 806 can be utilized by a cooling tower 808.

Moreover, as mentioned, FIG. 8 also illustrates an exemplary process for producing a comestible cell-based food product (e.g., as further described in relation to FIGS. 1 and 10A-10D) utilizing superheated water, process steam, and process fluid stored at a plurality of temperatures. For instance, as described above (e.g., in relation to FIG. 2), process equipment can be prepared for receiving cell culture media by cleaning-in-place (CIP) and sterilization-in-place (SIP) procedures. As shown in FIG. 8, cleaning-in-place (CIP) can include an act 812 utilizing a caustic solution and/or heated by steam, heated by process fluid from the stratified energy storage tank 806, or received directly from the stratified energy storage tank 806. Also, cleaning-in-place can include cold or ambient temperature processes at an act 814. Following cleaning-in-place, the process equipment of the bioreactor system can undergo sterilization-in-place utilizing superheated water from the SHW storage tank 804 as further described above (e.g., in relation to FIGS. 5 and 7).

As further shown in FIG. 8, in response to cleaning-in-place at acts 812 and 814 and sterilization-in-place at act 816, the system 800 introduces media powders 818 and treated water 820 into a mixer at act 822, wherein the treated water is brought to a selected temperature utilizing process fluid from a lower tier of the stratified energy storage tank 806. The mixed media powders 818 and treated water 820 can be further treated (e.g., pasteurized) by a high-temperature short time (HTST) procedure at act 824, with heat provided by an upper tier of the stratified energy storage tank 806. Further, the mixture can undergo a relatively colder HTST procedure at an act 826 to bring the mixture back to a moderate temperature, with cooling provided by a lower tier of the stratified energy storage tank 806.

Moreover, in response to preparing and treating the mixture of media powders 818 and treated water 820, at least one bioreactor can be seeded at an act 828 and cultivated at an act 830 to grow meat cells therein. The cultivated meat cells can then be cooled at an act 832, with cooling provided by a lower tier of the stratified energy storage tank 806, in preparation for holding at an act 834 and separation at an act 836. As also shown, additional cold process fluid can be utilized from a lower tier of the stratified energy storage tank 806 to drain or recycle excess material at an act 838.

Following separation of meat cells at the act 836, cells can be collected at an act 840 and media separated therefrom at an act 842 utilizing a wash water 844 provided directly from the stratified energy storage tank 806 (or cooled thereby). Then, a second round of separation at an act 846 and additional process fluid can be utilized from the stratified energy storage tank 806 to drain or recycle excess material at an act 848. Finally, at an act 852, a comestible cell-based food product 850 can be made ready for food processing, packaging, and consumption. As mentioned, additional details related to the foregoing procedures for producing comestible cell-based food products are provided in relation to FIGS. 10A-10D below.

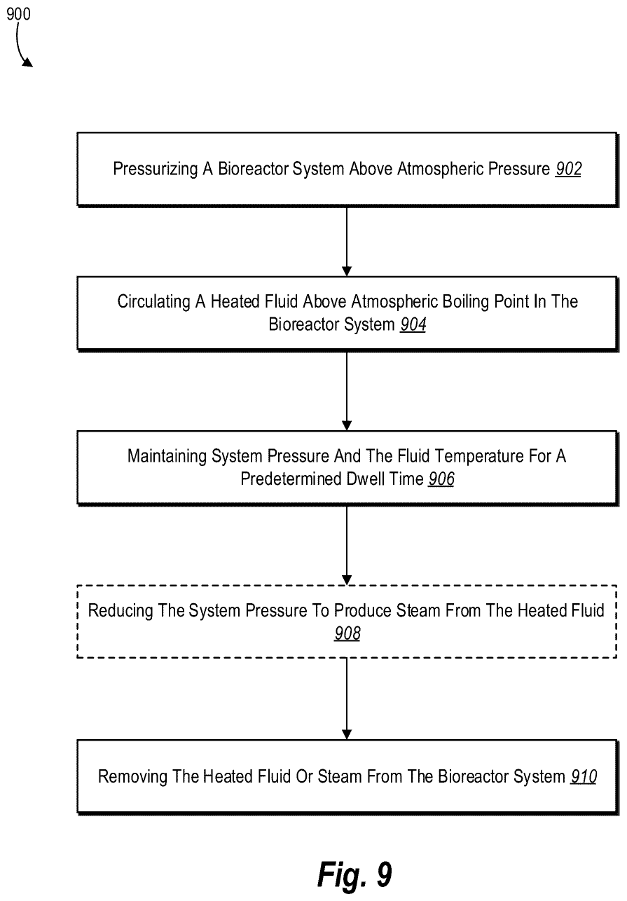

FIGS. 1-8, the corresponding text, and the examples provide several different systems, methods, techniques, components, and/or devices relating to sterilizing process equipment of bioreactor systems in accordance with one or more implementations. In addition to the above description, one or more implementations can also be described in terms of flowcharts including acts for accomplishing a particular result. FIG. 9, for example, illustrates such a flowchart of acts. The acts described herein may be repeated or performed in parallel with one another or in parallel with different instances of the same or similar acts.

FIG. 9 illustrates a series of acts 900 comprising an act 902 of pressurizing a bioreactor system above atmospheric pressure, an act 904 of circulating a heated fluid above atmospheric boiling point in the bioreactor system, an act 906 of maintaining the system pressure and the heated fluid temperature for a predetermined dwell time, an optional act 908 of reducing the system pressure to produce steam from the heated fluid, and an act 910 of removing the heated fluid or steam (depending on whether act 908 was performed to produce steam) from the bioreactor system.

The series of acts 900 comprises the act 902 of pressurizing a bioreactor system above atmospheric pressure. In particular, the act 902 comprises pressurizing at least one pressure vessel of a bioreactor system at a pressure above an atmospheric pressure.

The series of acts 900 includes the act 904 of circulating a heated fluid above atmospheric boiling point in the bioreactor system. In particular, the act 904 comprises circulating heated fluid in the bioreactor system, the heated fluid having a temperature above an atmospheric boiling point of the heated fluid.

The series of acts 900 also includes the act 906 of maintaining the system pressure and the heated fluid temperature for a predetermined dwell time. In particular, the act 906 comprises maintaining, in the at least one pressure vessel of the bioreactor system, the pressure above the atmospheric pressure and the temperature above the atmospheric boiling point for a predetermined dwell time.

In some embodiments, the series of acts 900 further includes the act 908 of reducing the system pressure to produce steam from the heated fluid within the bioreactor system. In particular, the act 908 comprises causing the heated fluid to evaporate within the at least one pressure vessel by reducing the pressure within the at least one pressure vessel.

The series of acts 900 also includes the act 910 of removing the heated fluid or steam from the bioreactor system. In particular, in embodiments wherein the act 908 is implemented to produce steam from the heated fluid, the act 910 includes removing the steam from within the at least one pressure vessel. Alternatively, in embodiments where in the act 908 is not implemented, the act 910 includes removing the heated fluid from the at least one pressure vessel.

In some implementations, the series of acts 900 further comprises pressurizing the at least one pressure vessel of the bioreactor system between 2 bar and 4 bar and circulating the heated fluid in the bioreactor system with the temperature between 110 degrees Celsius and 180 degrees Celsius.

In some implementations, the series of acts 900 further comprises modifying a chemical composition of the heated fluid with a mineral buffer.

In some implementations, the series of acts 900 further comprises adjusting an alkalinity of the heated fluid to a pH greater than 7.

In some implementations, the series of acts 900 further comprises intermittently reducing the pressure within the at least one pressure vessel during the predetermined dwell time to produce steam from the heated fluid.

In some implementations, the series of acts 900 further comprises maintaining the temperature of the heated fluid in the at least one pressure vessel during the predetermined dwell time by injecting additional heated fluid into the bioreactor system from a heated fluid source integrated with the bioreactor system.

In some implementations, the series of acts 900 further comprises pressurizing the at least one pressure vessel of the bioreactor system by forcing compressed air into the bioreactor system as the heated fluid is circulated through the bioreactor system.

In some implementations, the series of acts 900 further comprises circulating the heated fluid in the bioreactor system from a superheated water storage tank integrated with the bioreactor system.

The paragraphs above describe methods for forming a cell mass into a cell-based-food product. FIGS. 10A-10D and the following accompanying paragraphs describe procurement of cells and growth of cells into a cell mass in accordance with one or more implementations. Generally, FIGS. 10A-10D illustrate a process of collecting cells from an animal, growing cells in a favorable environment, banking successful cells, and collecting cells into a cell mass followed by de-wetting and/or other treatments.

As illustrated by step 1002 in FIG. 10A, tissue is collected from a living animal via biopsy. In particular, stem cells, mesenchymal progeny, ectoderm lineage, and/or endoderm lineages can be isolated from the removed tissue. In some implementations of the present disclosure, tissue, such as fat and others, are processed to isolate stem cells, mesenchymal, ectoderm, and/or endoderm progeny or lineage cells. As illustrated, tissue 1004 is removed from an animal. In some examples, the tissue 1004 is removed from a living animal by taking a skin sample from the living animal. For instance, skin or muscle samples may be taken from a chicken, cow, fish, shellfish or another animal.

Cells may be extracted from the tissue 1004 that was removed from the animal. More specifically, the tissue 1004 is broken down by enzymatic and/or mechanical means. To illustrate, FIG. 10A includes digested tissue 1006 that comprises the cells to be grown in cultivation.

Cells in the digested tissue 1006 may be proliferated under appropriate conditions to begin a primary culture. As illustrated in FIG. 10A, cells 1008 from the digested tissue 1006 are spread on a surface or substrate and proliferated until they reach confluence. As shown in FIG. 10A, in some cases, cells 1012 have reached confluence when they start contacting other cells in the vessel, and/or have occupied all the available surface or substrate.

In some examples, cells are stored and frozen (i.e., banked) at different steps along the cell culture process. Cryopreservation generally comprises freezing cells for preservation and long-term storage. In some implementations, tissue and/or cells are removed from a surface or substrate, centrifuged to remove moisture content, and treated with a protective agent for cryopreservation. For example, as part of cryopreservation, tissues and cells are stored at temperatures at or below-80C. The protective agent may comprise dimethyl sulfoxide (DMSO) or glycerol.

Cells stored through cryopreservation may be used to replenish working cell stock. For instance, while a portion of the digested tissue 1006 is used as the cells 1008 spread on a surface or substrate, the remaining or excess digested tissue 1006 is transferred to cryovials 1010 for storage. Furthermore, the cells 1012 may be banked once reaching confluence and stored in cryovials 1014.

Once the cells 1012 have reached confluence, or just before the cells 1012 have reached confluence (e.g., occupation of about 80% of the substrate), the disclosed process comprises a series of cell passage steps. During cell passage, the cells 1012 are divided into one or more new culture vessels for continued proliferation. To illustrate, the cells 1012 may be diluted or spread on one or more surfaces or substrates to form the cells 1018. The cells 1018 are then grown 1016 to confluence, or just before confluence.

The cycle of dividing the cells 1012 into the cells 1018 for continued proliferation in new culture vessels may be repeated for a determined number of cycles. Typically, cell lines derived from primary cultures have a finite life span. Passaging the cells allows cells with the highest growth capacity to predominate. In one example, cells are passaged for five cycles to meet a desired genotypic and phenotypic uniformity in the cell population.

In some implementations, the disclosed method comprises immortalizing cells that have been grown and passaged for the determined number of cycles. For instance, the cells 1018 may be immortalized. As shown in FIG. 10B, cells 1020 have demonstrated a preferred growth capacity to proceed to immortalization. To achieve immortalization, the disclosed process transfects the cells 1020 with genes of interest. In one example telomerase reverse transcriptase (TERT) is introduced to the cells 1020. In some implementations, the cells may be subjected to a selection process as known by those skilled in the art. The cells 1020 may then be passaged for a predetermined set of passaging cycles. In one example passaging cycle, the cells 1020 are grown to (or near) confluence 1024, then they are reseeded in new growth vessels, preserved in vials 1022, or some combination of both. The disclosed process may include any number of passaging cycles to ensure that the cells have reached immortality (e.g., can passage 60+ times without senescing), a target growth capacity, and/or a target quantity for banking. For example, cells may be passaged until they have reached a passage level of 100 (e.g., have been passaged for 100 passaging cycles). In another example, cells are passaged until they reach a population doubling level of 100.

Cells that have reached immortality or a target growth capacity by living through a target passage level may be adapted to suspension culture. In one example, a suspension culture media and agitation of cells in this suspension environment help cells to adapt and start proliferating in the new growth environment. The cells adapted to suspension 1026 may be stored in cryovials 1028 for cryopreservation and banking. Cells in suspension 1026 will begin to proliferate and the process begins a series of dilute and expand steps.

During dilution and expansion, cells are moved from growth vessels into newer, and progressively larger, growth vessels. For example, cells in suspension 1026 may begin in a single tube. The cells will proliferate and increase in cellular density. Once the cells have reached a target cell number (i.e., viable cell density (VCD) at desired volume), they are diluted and moved to a larger growth vessel. Optionally, the cells are banked in cryovials throughout expansion. For example, once cells in suspension reach a maximum VCD, the cells may begin to leave exponential growth due to overcrowding. After reaching a target density, the suspension cells may be transferred to a larger vessel 1030 and diluted with additional media. The dilute-and-expand steps are repeated using progressively larger vessels (e.g., the vessel 1031 and the vessel 1032) and/or progressive dilution until the cells reach a production-ready volume. For example, cells may be production ready at about a 1,000-100,000 liter scale at 5 million cells per mL. The cells may be banked in cryovials at any of the dilution and expansion cycles.

As part of preparing cells to form cell-based food products, the disclosed process comprises growing the cells as an adherent culture. Generally, cells that are grown attached to a substrate form a texture that more closely resembles tissue found in conventional meat. Thus, the cells may be transferred from growth in suspension to growth in an adherent reactor. For example, the cells grown in suspension in the vessel 1032 may be transferred to growth on a substrate. FIG. 10C illustrates a bioreactor system comprising a plurality of adherent bioreactors 1048 connecting in parallel to a media vessel 1040. The media vessel 1040 holds the cells grown in suspension media. In some implementations, cells from the vessel 1032 are transferred directly to a cell culture media (or just “media”) vessel 1040. In one example, the media vessel 1040 comprises the vessel 1032. The adherent bioreactors 1048 may comprise pipe-based bioreactors. As shown, a plurality of valves 1044 is secured to the plurality of adherent bioreactors 1048 to enable individual use and access of each of the adherent bioreactors 1048. For instance, to limit flow to only a first bioreactor of the plurality of adherent bioreactors 1048, the valve 1044 of the first bioreactor is opened while the remaining valves 1044 are closed. Furthermore, the bioreactor system can include a directional valve 1042 for changing between flow directions.

In some implementations, and as illustrated in FIG. 10C, cells (e.g., adherent cells or suspension adapted cells) are prepared by flowing cells suspended in media (e.g., cell culture media) across substrates in the plurality of adherent bioreactors 1048. More particularly, cells from the media vessel 1040 may contact or land on the substrates in the plurality of adherent bioreactors 1048. Cells and media that flowed through the adherent bioreactors 1048 are cycled back to the media vessel 1040. The media and cells can be cycled through the adherent bioreactors 1048 until a target adherent cell density is reached. For instance, in some implementations, the disclosed method comprises measuring a cell density of outflow from the adherent bioreactors 1048 to infer an adherent cell density.

The cells grow into adherent tissue within the adherent bioreactors 1048. Once they have grown to a target density, either according to a learned timing or according to a measured fluctuation in cell metabolism of components such as glucose and oxygen, then the adherent tissue is ready for removal. The removal process of the disclosed method uses a high-pressure flow to shear the adherent tissue off the substrate surfaces. In one example, wash buffer from a wash tank 1056 is flowed across the substrates in the adherent bioreactors 1048. The wash buffer and cell mixture are flowed through a filter 1052 where the cells are collected into one or more cell masses 1054.

The cell masses 1054 may be further processed to adjust moisture content. FIG. 10D illustrates an example apparatus for reducing moisture content in the cells. In particular, FIG. 10D illustrates a pressure apparatus 1060 that compresses the cell masses 1058a and 1058b. While FIG. 10D illustrates a mechanical method for adjusting moisture content of the cell masses 1058a and 1058b, other methods may be used to adjust moisture content. For example, the cell masses 1058a and 1058b may be mixed with a drying agent, vacuum dried, centrifuged, or otherwise dried. A moisture-adjusted-cell mass may be transferred to a container 1062 for additional processing. For example, the cell mass 1058a or 1058b may be removed from the container 1062 to be formed into a cell-based food product.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various implementations of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to implementations containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to implementations containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art and are to be construed as being without limitation to such specifically recited examples and conditions. Although implementations of the present disclosure have been described in detail, it should be understood that the various changes, substitutions, and alterations could be made hereto without departing from the spirit and scope of the present disclosure.

The present invention may be embodied in other specific forms without departing from its spirit or essential characteristics. Indeed, the described implementations are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A method for sterilizing process equipment configured to produce comestible cell-based food products, the method comprising:
pressurizing at least one pressure vessel of a bioreactor system at a pressure above an atmospheric pressure;
circulating heated fluid in the bioreactor system, the heated fluid having a temperature above an atmospheric boiling point of the heated fluid;
maintaining, in the at least one pressure vessel of the bioreactor system, the pressure above the atmospheric pressure and the temperature above the atmospheric boiling point for a predetermined dwell time; and
removing the heated fluid from the at least one pressure vessel.

2. The method of claim 1, further comprising:
pressurizing the at least one pressure vessel of the bioreactor system between 2 bar and 4 bar; and
circulating the heated fluid in the bioreactor system with the temperature between 110 degrees Celsius and 180 degrees Celsius.

3. The method of claim 1, further comprising modifying a chemical composition of the heated fluid with a mineral buffer.

4. The method of claim 1, further comprising adjusting an alkalinity of the heated fluid to a pH greater than 7.

5. The method of claim 1, further comprising causing the heated fluid to evaporate within the at least one pressure vessel by reducing the pressure within the at least one pressure vessel.

6. The method of claim 1, further comprising maintaining the temperature of the heated fluid in the at least one pressure vessel during the predetermined dwell time by injecting additional heated fluid into the bioreactor system from a heated fluid source integrated with the bioreactor system.

7. The method of claim 1, further comprising pressurizing the at least one pressure vessel of the bioreactor system by forcing compressed air into the bioreactor system as the heated fluid is circulated through the bioreactor system.

8. The method of claim 1, further comprising circulating the heated fluid in the bioreactor system from a superheated water storage tank integrated with the bioreactor system.

9. A bioreactor system for preparing a comestible cell-based food product, the bioreactor system comprising:
at least one bioreactor configured to grow the comestible cell-based food product;
an integrated superheated water source configured to provide, to the at least one bioreactor, heated fluid having a pressure above an atmospheric pressure and a temperature above an atmospheric boiling point of the heated fluid; and
a compressor configured to pressurize the bioreactor system as the heated fluid is circulated in the at least one bioreactor.

10. The bioreactor system of claim 9, wherein the integrated superheated water source comprises a superheated water storage tank configured to store the heated fluid at the pressure above the atmospheric pressure and at the temperature above the atmospheric boiling point for the heated fluid.

11. The bioreactor system of claim 9, wherein the integrated superheated water source comprises a superheated water generator comprising a direct-heated hot water boiler configured to generate the heated fluid at the pressure above the atmospheric pressure and at the temperature above the atmospheric boiling point for the heated fluid.

12. The bioreactor system of claim 9, further comprising an integrated process steam generator configured to circulate process steam in the bioreactor system.

13. The bioreactor system of claim 12, wherein the integrated superheated water source comprises a superheated water generator comprising a hot water boiler configured to generate, utilizing process steam from the integrated process steam generator to heat a process fluid, the heated fluid at the pressure above the atmospheric pressure and at the temperature above the atmospheric boiling point for the heated fluid.

14. The bioreactor system of claim 9, further comprising one or more integrated energy storage tanks configured to store process fluid at one or more process temperatures and to provide the process fluid to one or more components of the bioreactor system.

15. An apparatus configured to simultaneously produce process steam and superheated water, the apparatus comprising:
an insulated enclosure comprising:
a lower boiler section configured to produce process steam; and
a superheated water generator disposed above the lower boiler section within the insulated enclosure, the superheated water generator comprising a heat exchanger configured to transfer heat from the process steam to fluid within the superheated water generator.

16. The apparatus of claim 15, further comprising one or more heating elements disposed below the heat exchanger of the superheated water generator, the one or more heating elements configured to convert condensate falling from the heat exchanger into additional process steam.

17. The apparatus of claim 16, wherein the heat exchanger of the superheated water generator comprises a pressurized enclosure configured to store the fluid within the superheated water generator at a pressure above an atmospheric pressure.

18. The apparatus of claim 17, further comprising an insulated storage tank configured to store fluid output from the superheated water generator at the pressure above the atmospheric pressure and at a temperature above an atmospheric boiling point for the fluid.

19. The apparatus of claim 15, wherein the fluid within the superheated water generator comprises water treated with one or more of a mineral buffer or an alkalinity buffer.

20. The apparatus of claim 15, further comprising a steam outlet valve for releasing process steam into an integrated system for cleaning and sterilizing process equipment.
